(19) United States
(12) Patent Application Publication
(10) Pub. No.: US 2018/0118806 A1
(43) Pub. Date: May 3, 2018

(54) ENGINEERED REPLACEMENT OF PARTIAL VARIANT WATER SOLUBLE MEMBRANE PROTEINS

(71) Applicant: Massachusetts Institute of Technology, Cambridge, MA (US)

(72) Inventor: Shuguang Zhang, Lexington, MA (US)

(21) Appl. No.: 15/657,650

(22) Filed: Jul. 24, 2017

Related U.S. Application Data
(60) Provisional application No. 62/366,306, filed on Jul. 25, 2016.

(51) Int. Cl. C07K 14/715

(52) U.S. Cl. CPC C07K 14/7158

(57) ABSTRACT

The present invention is directed to water-soluble membrane proteins, methods for the preparation thereof and methods of use thereof.

### REFERENCE TO RELATED APPLICATIONS

This application claims the benefit of the filing date under 35 U.S.C. § 119(e) to U.S. Provisional Patent Application No. 62/366,306, filed on Jul. 25, 2016, the entire contents of which, including all sequences and drawings, are incorporated herein by reference.

This application is also related to U.S. Ser. No. 13/403,725, filed on Feb. 23, 2012, now U.S. Pat. No. 8,637,452; U.S. Ser. No. 14/105,252, filed on Dec. 13, 2013, now U.S. Pat. No. 9,309,302; U.S. Ser. No. 14/669,753, filed on Mar. 26, 2015, now published as US 2015-0370960 A1; and U.S. Ser. No. 14/723,399, filed on May 27, 2015, now published as US 2015-0370961 A1. Each of the above referenced patents and applications are hereby incorporated herein by reference.

### SEQUENCE LISTING

The application contains a Sequence Listing which has been submitted electronically in ASCII format and is hereby incorporated by reference in its entirety. Said ASCII copy, created on Oct. 12, 2017, is named 122288-09302_SL.txt and is 37,388 bytes in size.

### BACKGROUND OF THE INVENTION

Membrane proteins are of critical importance in understanding biological systems. The development of techniques for making modified water soluble polypeptides, for example, in which certain hydrophobic residues replace hydrophilic residues can be utilized for various applications.

A critical and challenging task is that it is extremely difficult to produce milligram quantities of soluble and stable receptors. Inexpensive large-scale production methods are desperately needed. It is only possible to conduct detailed structural studies once these preliminary obstacles have been surmounted. Therefore, there is a need in the art for improved methods of making G-protein coupled receptors.

### SUMMARY OF THE INVENTION

The present invention is directed to variant water-soluble membrane peptides, compositions comprising said peptides, methods for the preparation thereof and methods of use thereof.

In one aspect, the invention provides a variant water-soluble polypeptide comprising a modified transmembrane (TM) α-helical domain, wherein the modified TM α-helical domain comprises an amino acid sequence in which one or more hydrophobic amino acid residues is replaced by one or more hydrophilic amino acid residues according to, for example, the QTY code method described in U.S. Pat. Nos. 8,637,452, 9,309,302, and US-2015-0370961-A1, the entire contents of which is incorporated herein by reference; and wherein a domain of the water-soluble polypeptide involved in ligand binding is absent.

Another aspect of the invention provides a method of preparing a variant water-soluble polypeptide, the method comprising replacing one or more hydrophobic amino acid residues within an α-helical TM domain of a native membrane protein with one or more hydrophilic amino acid residues according to, for example, the QTY code method described in U.S. Pat. Nos. 8,637,452, 9,309,302, and US-2015-0370961-A1, the entire contents of which is incorporated herein by reference; and wherein a domain of the water-soluble polypeptide involved in ligand binding is absent.

Yet another aspect of the invention provides a variant polypeptide prepared by replacing one or more hydrophobic amino acid residues within an α-helical TM domain of a native membrane protein with one or more hydrophilic amino acid residues, wherein a domain of the water-soluble polypeptide involved in ligand binding is absent.

In certain embodiments, the membrane protein/polypeptide is an integral membrane protein. In certain embodiments, the membrane protein/polypeptide is a mammalian protein. For example, the membrane protein may be an olfactory receptor, such as mOR103-15.

In certain embodiments, the α-helical domain is a 7-transmembrane α-helical domain.

In certain embodiments, the variant is a water-soluble variant of a G-protein coupled receptor (GPCR), having seven transmembrane α-helical domains (TM1-TM7), linked by 4 extracellular loops (EC1-EC4) and 3 intracellular loops (IC1-IC3), and flanked by an extracellular N-terminal sequence and an intracellular C-terminal sequence.

In certain embodiments, one or more phenylalanine (F) residues of the α-helical domain of the protein are replaced with tyrosine (Y). In certain additional embodiments, one or more isoleucine (I) and/or valine (V) residues of the α-helical domain of the protein are replaced with threonine (T). In yet additional aspects, one or more phenylalanine residues of the α-helical domain of the protein are replaced with tyrosine and one or more isoleucine and/or valine residues of the α-helical domain of the protein are replaced with threonine. In additional embodiments, one or more leucine (L) residues of the α-helical domain of the protein are replaced with glutamine (Q) or asparagine (N). In yet additional embodiments, one or more leucine residues of the α-helical domain of the protein are replaced with glutamine/asparagine and one or more isoleucine and/or valine residues of the protein are replaced with threonine. In further embodiments, one or more leucine residues of the α-helical domain of the protein are replaced with glutamine/asparagine and one or more phenylalanine residues of the α-helical domain of the protein are replaced with tyrosine. In yet additional aspects, one or more leucine residues of the α-helical domain of the protein are replaced with glutamine/asparagine, one or more phenylalanine residues of the α-helical domain of the protein are replaced with tyrosine, and one or more isoleucine and/or valine residues of the α-helical domain of the protein are replaced with threonine.

In certain embodiments, one or more amino acids within potential ligand binding sites of the native membrane protein are not replaced.

In certain embodiments, in said variant: (1) 7-transmembrane α-helical hydrophobic residues Leucine (L), isoleucine (I), valine (V), and phenylalanine (F) in hydrophilic surface α-helical positions b, c, and f but not positions a, d, e, and g of the GPCR have been substituted by glutamine (Q) or Asparagine (N), threonine (T), threonine (T), and tyrosine (Y), respectively, and, (2) a domain of the GPCR, or a portion of the domain, is absent, wherein said domain is selected from the group consisting of: the N-terminal extracellular sequence, a 7-transmembrane α-helical domain, and an extracellular loop (EC).

In certain embodiments, the variant comprises the full length or a partial N-terminal extracellular sequence.

In certain embodiments, a 7-transmembrane α-helical domain is absent. For example, in certain embodiments, two or more 7-transmembrane α-helical domains are absent. In certain embodiments, two or more consecutive 7-transmembrane α-helical domains are absent. In certain embodiments, two or more non-consecutive 7-transmembrane α-helical domains are absent.

In certain embodiments, an EC is absent. For example, in certain embodiments, two or more ECs are absent. In certain embodiments, two or more consecutive ECs are absent. In certain embodiments, two or more non-consecutive ECs are absent.

In certain embodiments, an IC is absent. For example, in certain embodiments, two or more ICs are absent. In certain embodiments, two or more consecutive ICs are absent. In certain embodiments, two or more non-consecutive ICs are absent.

In certain embodiments, the variant has reduced ligand binding affinity as compared to the wildtype GPCR. For example, the ligand binding affinity of the variant may be 90%, 80%, 70%, 60%, 50%, 40%, 30%, 20%, 10%, 5% or less of that of the native TM protein.

In certain embodiments, the variant has reduced ligand binding specificity as compared to the wildtype GPCR from which the variant is derived.

In certain embodiments, the variant has a biological activity of the GPCR. For example, in certain embodiments, the variant retains the ligand-binding activity of the native membrane protein (GPCR)—i.e., the biological activity is ligand binding. In certain embodiments, the biological activity is at least substantially similar binding affinity for a native ligand of the GPCR.

In certain embodiments, the pI of the variant is substantially the same as the pI of the GPCR.

In certain embodiments, the variant comprises conservative substitutions at other parts of the variant.

In certain embodiments, at least 25 said 7-transmembrane α-helical hydrophobic residues L, I, V, and F are replaced.

In certain embodiments, the GPCR is a mammalian receptor.

In certain embodiments, the GPCR is selected from the group consisting of: purinergic receptors (P2Y₁, P2Y₂, P2Y₄, P2Y₆), M₁ and M₃ muscarinic acetylcholine receptors, receptors for thrombin [protease-activated receptor (PAR)-1, PAR-2], thromboxane (TXA₂), sphingosine 1-phosphate (S1P₂, S1P₃, S1P₄ and S1P₅), lysophosphatidic acid (LPA₁, LPA₂, LPA₃), angiotensin II (AT₁), serotonin (5-HT₂, and 5-HT₄), somatostatin (sst₅), endothelin (ET_{A} and ET_{B}), cholecystokinin (CCK₁), V₁ₐ vasopressin receptors, D₅ dopamine receptors, fMLP formyl peptide receptors, GAL₂ galanin receptors, EP₃ prostanoid receptors, A₁ adenosine receptors, α₁ adrenergic receptors, BB₂ bombesin receptors, B₂ bradykinin receptors, calcium-sensing receptors, chemokine receptors, KSHV-ORF74 chemokine receptors, NK₁ tachykinin receptors, thyroid-stimulating hormone (TSH) receptors, protease-activated receptors, neuropeptide receptors, adenosine A2B receptors, P2Y purinoceptors, metabolic glutamate receptors, GRK5, GPCR-30, CCR5, and CXCR4. In certain embodiments, the GPCR is a CXCR4 or CCR5.

In certain aspects of the invention, the secondary structure of the water-soluble peptide is determined. In some embodiments, the secondary structure is determined using circular dichroism.

In certain embodiments, ligand binding to the water-soluble polypeptide is measured. In some aspects, ligand binding affinity of the water-soluble polypeptide is compared to that of the native protein. In additional aspects, ligand binding is measured using microscale thermophoresis (MST), calcium influx assay, or any combination thereof.

In a further aspect, the invention provides a method of treatment for a disorder or disease that is mediated by the activity a membrane protein in a subject in need thereof, comprising administering to said subject an effective amount of the subject water-soluble polypeptide comprising a modified α-helical domain.

Examples of disorders and diseases that can be treated by administering a water-soluble peptide of the invention include, but are not limited to, cancer (such as, small cell lung cancer, melanoma, triple negative breast cancer), Parkinson's disease, cardiovascular disease, hypertension, and bronchial asthma.

The invention also provides a pharmaceutical composition comprising the subject water-soluble polypeptide of the invention and pharmaceutically acceptable carrier or diluent.

In yet another aspect, the invention provides a cell transfected with a subject water-soluble peptide comprising a modified α-helical domain. In certain embodiments, the cell is a mammalian cell.

It should be understood that any one embodiment of the invention can be combined with any other embodiment, including those only described in the examples or under one aspect of the invention, unless explicitly disclaimed or otherwise inappropriate.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-1D show schematic drawings (not necessarily to scale) that illustrate certain non-limiting embodiments of the subject partial water-soluble variant (“partial variant”) of a G-protein coupled receptor (GPCR).

FIG. 1A shows the binding between a wildtype (wt) GPCR and its ligand—the natural GPCR has 4 bind sites, N-terminus and each of the external loop bind to different part of the peptide or protein ligand. For illustrative purpose only, the binding between the GPCR extracellular loops (ECs) and the ligand is depicted as consecutive matching pairs of shapes of protrusions and indentations, while no such limitation is intended. For clarity, the plasma membrane is not shown.

FIG. 1B shows one illustrative embodiment of the subject partial variants, in which TM4 (the 4^{th} transmembrane domain) and beyond of the GPCR is absent due to a C-terminal truncation. It purportedly only uses its N-terminus and the first external loop to bind to its ligand, thus occupying 2 biding sites, perhaps with reduced affinity.

FIG. 1C shows one illustrative embodiment of the subject partial variants, in which TM3-TM6 and IC2-IC3 are absent, while the remaining portion of the GPCR, including all ECs, are present. It purportedly uses the N-terminus and its 3 external loops to bind to the natural ligand. Also maybe present is/are optional artificial linker sequence(s) that connect(s) remaining segments or domains of the GPCR that are otherwise not directly linked in wt GPCR, such as EC1 and EC2, and EC2 and EC3.

FIG. 1D shows one illustrative embodiment of the subject partial variants, in which TM4-TMS, and IC2 and IC3 are fused. It may bind its ligand with reduced affinity.

FIG. 2 shows two representative partial variants of the invention—SZ218a (SEQ ID NO: 9) and SZ190b (SEQ ID NO: 6)—both derived from the full-length QTY variant of CCR5 (CCR5^{QTY}). Binding specificity and gene activation activity of the variants are also indicated.

FIG. 3 shows three representative partial variants of the invention—SZ352a (SEQ ID NO: 10), SZ218a (SEQ ID NO: 9) and SZ190b (SEQ ID NO: 6)—all derived from the full-length QTY variant of CCR5 (CCR5^{QTY}). Binding specificity to natural ligand Rantes (CCL5) and gene activation activity of the variants are also indicated.

FIG. 4 shows Rantes (CCL5) ligand binding to non-full length CXCR4^{QTY} variants (SEQ ID NOS: 11-17, 17, 17-21, and 21-22, respectively, in order of appearance).

FIG. 5 illustrates the binding of full-length (352 aa) CCR5^{QTY} variant to its natural ligand Rantes (CCL5), as measured by fluorescent MST (MicroScale Thermophoresis) binding assay. The measured K_{d} is about 30 nM. Tech. 1 and Tech. 2 stand for Technical Repeat 1 and 2, respectively. In the binding curves of the full-length CCR5^{QTY} variant with Rantes, each red and blue dot represents a different Rantes concentration. The red and blue lines are the data fittings. The K_{d} value is derived from 50% of the middle point between.

FIG. 6 illustrates the binding of non-full length CCR5^{QTY} (218 aa) to its natural ligand Rantes. The K_{d} is about 80 nM.

FIG. 7 illustrates the binding of non-full length CCR5^{QTY} (190 aa) to its natural ligand Rantes. The K_{d} is about 80 nM. The K_{d} is about 46-48 nM.

### DETAILED DESCRIPTION OF THE INVENTION

It is generally believed that natural proteins require full length to be completely functional. Presumably, the deletion of large parts of proteins, especially membrane proteins, would disable their ligand-binding activities, rendering them no longer functional. Through bioinformatics analysis, numerous pseudo genes with large deletions and truncations exist in the genomes that were assumed to code for non-functional proteins.

However, results described herein, obtained by using large libraries of water-soluble detergent free membrane proteins (constructed by a method referred to as “QTY Code,” see U.S. Pat. Nos. 8,637,452 and 9,309,302, both incorporated herein by reference), has yielded a surprising result.

Using the yeast 2-hybrid system to screen over 2 million engineered water-soluble variants of CXCR4, CCR3, CCR5, and CX3CR1, followed by stringent yeast mating selection, receptor-ligand interaction for gene activation of G protein-coupled chemokine receptors CXCR4 and CCR5 were selected. Surprisingly, a subset of partial or Non-Full length CCR5^{QTY} (nfCCR5^{QTY}) and Non-Full length CXCR4^{QTY} (nfCXCR4^{QTY}) retained natural ligand-binding function. A candidate of nfCCR5^{QTY} named SZ218a, with only 218 (˜62%) of the 352 full-length amino acids, was expressed in SF9 insect cells and affinity-purified. The nfCCR5^{QTY} SZ218a variant showed that the non-full length receptor is able to bind its natural ligand Rantes of nfCCR5^{QTY} at the nanomolar range. The non-full length receptor possesses the N-terminus and parts of the 3^{rd} external cellular loop. Another partial variant derived from CXCR4^{QTY}, named SZ146a, has only 146 amino acids. This partial variant was expressed in cell-free system and affinity-purified. This partial variant can also bind to the natural ligand SDF1a. Subsequently, several additional non-full length GPCRs were also experimentally tested.

The non-full length/partial GPCR of the invention can be generated using routine/standard molecular biology techniques. For example, in certain embodiments, after generating the full length water-soluble variants of a GPCR using the QTY method (the full-length QTY variant), a library of partial sequences, each encompassing a modified QTY variant that has a different ligand-binding domain(s) being absent, can be readily generated, and the ability of those partial sequences to bind to the native ligand tested in vitro and/or in vivo. For example, the traditional yeast 2-hybrid system can be used to identify partial QTY variants having stronger binding affinity and/or specificity to the native ligand. In other embodiments, selected ligand binding domains or fragments thereof may be selected to construct nfGPCR with said selected ligand binding domains or fragments thereof. In doing so, a partial TM or EC region may be used. Additional artificial linker sequences may be included in the subject partial variant, since spacing between any newly connected ligand binding domains may be important to preserve binding affinity/specificity.

In certain embodiments, point mutations or small insertions or deletions may be further introduced into the variants to fine tune binding specificity and/or affinity to the natural ligand.

The non-full length receptors may be further engineered for a number of biotechnological, diagnostic and therapeutic applications including (but not limited to): 1) use to accelerate drug discovery and drug screening, similar as certain protein kinases were used for similar purposes, 2) use as trapping for decoy therapies, 3) use to couple Fc domain of antibodies such as IgG to the high-affinity non-full-length receptors, 4) use to generate vaccines using the QTY Code engineered viral membrane proteins, and 5) used as engineered diagnostic devices for detecting their native ligand concentration in a biological environment (in vitro or in vivo).

A description of preferred embodiments of the invention follows.

The words “a” or “an” are meant to encompass one or more, unless otherwise specified.

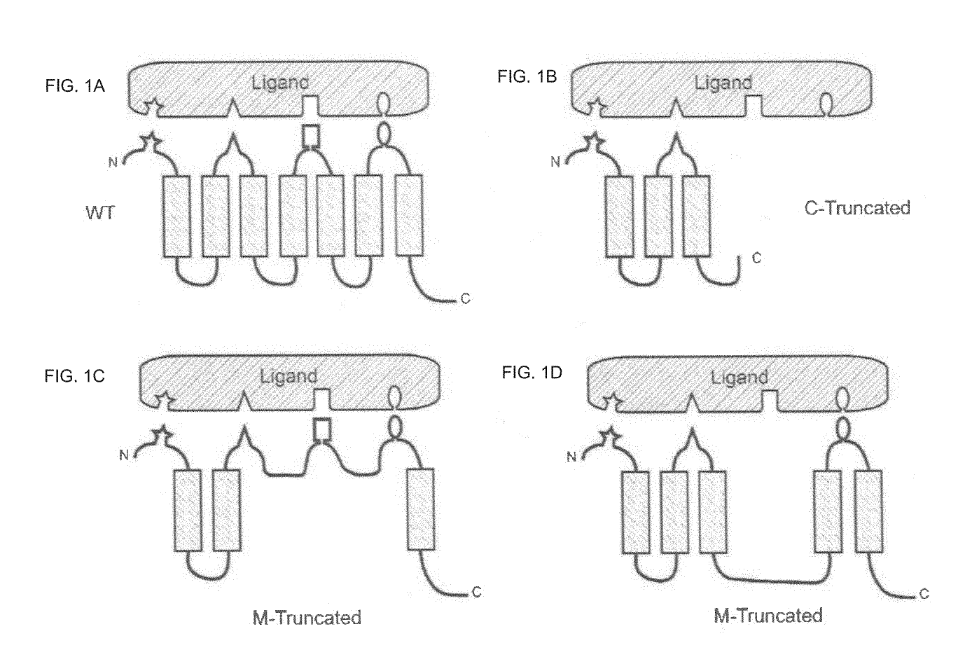

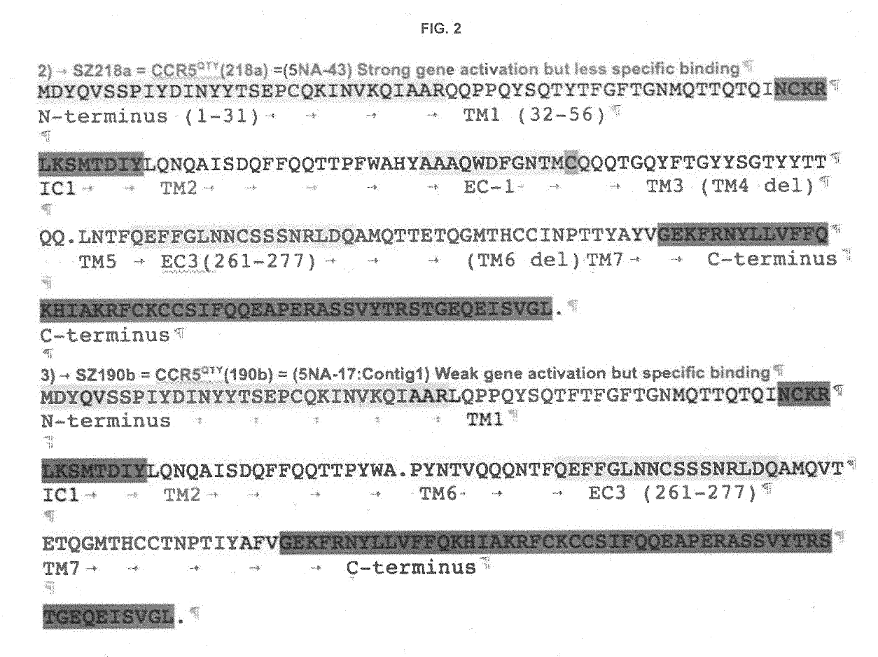

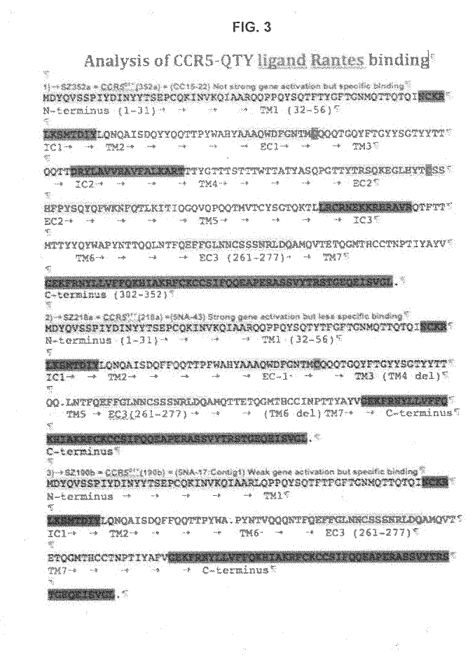

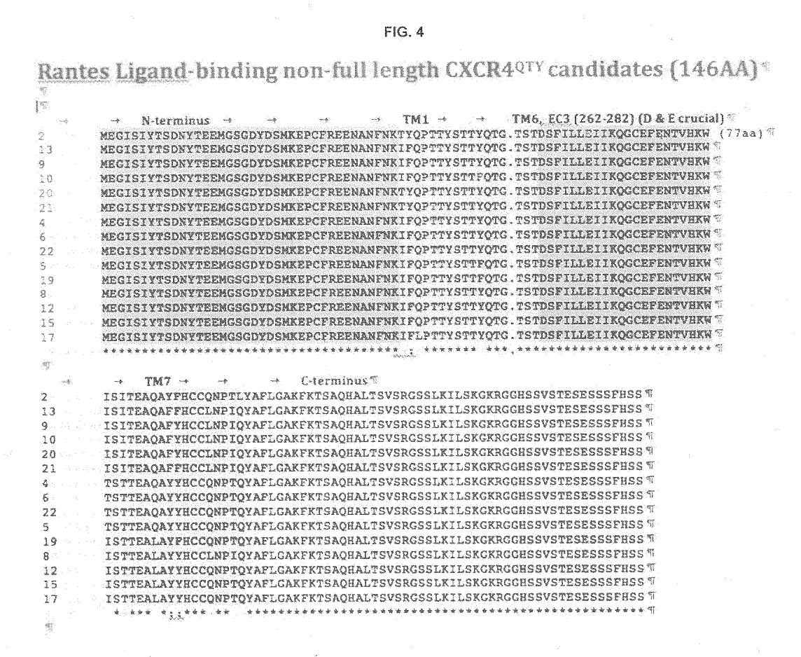

In some aspects, the invention is directed to the use of a replacement method to systematically change the 7-transmembrane α-helix hydrophobic residues of a native protein to hydrophilic residues. This invention converts the native membrane protein from a water-insoluble protein to a water-soluble formulation. Furthermore, the water-soluble variant does not need to be full-length, and may have one or more domains involved in ligand binding, such as the extracellular N-terminal sequence and/or one or more of the EC domains, absent. See FIGS. 1A-1D for a few representative embodiments of the partial variants of the invention. In certain embodiments, the extracellular N-terminal sequence is present in the non-full length/partial variant.

As described in detail in the related applications, now U.S. Pat. Nos. 8,637,452, 9,309,302, and US-2015-0370961-A1, the invention provides a method (the QTY method) to systematically and selectively change key residues at the α-helical positions b, c, f that usually face the hydrophilic surface, while maintaining the hydrophobic residues at α-helical positions a, d, e, g. The synthetic biology design method is general and broadly applicable to the study of other TM proteins such as G-protein coupled receptors.

The QTY replacement method is partly based on the α-helical forming tendencies of eight amino acids: leucine (L) (1.30), glutamine (Q) (1.27) or asparagine (N), phenylalanine (F) (1.07), tyrosine (Y) (0.72), isoleucine (I) (0.97), valine (V) (0.91) and threonine (T) (0.82). In addition, side chains of Q, Y and T can all form hydrogen bonds with water: Q can form 4H-bonds (2H-donors from —NH₂, 2 H-acceptors from C═O), and T and Y can form 3H-bonds each (—OH, 1-H donor from —H and 2 acceptors from —O). Thus the Q, T, Y residues are more water-soluble than L, F, I, or V, which cannot form any hydrogen bonds with their side chains. The QTY substitutions generally do not lead to positive- or negative-charge changes. Furthermore, the molecular shapes and sizes are very similar for the pairs: leucine/glutamine or asparagine, phenylalanine/tyrosine, valine/threonine, and isoleucine/threonine. The QTY changes should thus increase the solubility of 7-transmembrane α-helices while maintaining the overall helical structure.

In certain embodiments, after performing the following substitutions: phenylalanine to tyrosine (F->Y), isoleucine/valine to threonine (I/V->T), and leucine to glutamine or asparagine (L->Q/N), and after removing certain domains involved in ligand binding, the secondary structure of the water-soluble GPCR receptor, as well as its ligand-binding capabilities can be examined.

The secondary structure and binding of the designed water-soluble GPCR receptor with the native GPCR receptor can be prepared. Milligram quantities of the water-soluble receptor can be produced and crystal screens can be set up with and without receptor ligands.

In one embodiment, the native membrane protein is a G-protein coupled receptor (GPCR). In yet another embodiment, the native membrane protein is an olfactory receptor. In some embodiments, the GPCR or the olfactory receptor is a mammalian receptor. In certain embodiments, the GPCR is CCR5 or CXCR4. In yet another embodiment, the olfactory receptor is mOR103-15. In certain aspects, the water-soluble polypeptide retains at least some of the biological activity of the native membrane protein. In yet another aspect, the membrane protein is a membrane receptor that mediates a disease or condition.

In certain embodiments, the native membrane protein is a GPCR selected from the group consisting of: purinergic receptors (P2Y₁, P2Y₂, P2Y₄, P2Y₆), M₁ and M₃ muscarinic acetylcholine receptors, receptors for thrombin [protease-activated receptor (PAR)-1, PAR-2], thromboxane (TXA₂), sphingosine 1-phosphate (S1P₂, S1P₃, S1P₄ and S1P₅), lysophosphatidic acid (LPA₁, LPA₂, LPA₃), angiotensin II (AT₁), serotonin (5-HT₂, and 5-HT₄), somatostatin (sst₅), endothelin (ET_{A} and ET_{B}), cholecystokinin (CCK₁), V₁ₐ vasopressin receptors, D₅ dopamine receptors, fMLP formyl peptide receptors, GAL₂ galanin receptors, EP₃ prostanoid receptors, A₁ adenosine receptors, α₁ adrenergic receptors, BB₂ bombesin receptors, B₂ bradykinin receptors, calcium-sensing receptors, chemokine receptors, KSHV-ORF74 chemokine receptors, NK₁ tachykinin receptors, thyroid-stimulating hormone (TSH) receptors, protease-activated receptors, neuropeptide receptors, adenosine A2B receptors, P2Y purinoceptors, metabolic glutamate receptors, GRK5, GPCR-30, CCR5, and CXCR4.

In a further embodiment, the invention is directed to a pharmaceutical composition or method of treatment described herein wherein the native membrane protein is a GPCR selected from the group consisting of: purinergic receptors (P2Y₁, P2Y₂, P2Y₄, P2Y₆), M₁ and M₃ muscarinic acetylcholine receptors, receptors for thrombin [protease-activated receptor (PAR)-1, PAR-2], thromboxane (TXA₂), sphingosine 1-phosphate (S1P₂, S1P₃, S1P₄ and S1P₅), lysophosphatidic acid (LPA₁, LPA₂, LPA₃), angiotensin II (AT₁), serotonin (5-HT_{2c} and 5-HT₄), somatostatin (sst₅), endothelin (ET_{A} and ET_{B}), cholecystokinin (CCK₁), V₁ₐ vasopressin receptors, D₅ dopamine receptors, fMLP formyl peptide receptors, GAL₂ galanin receptors, EP₃ prostanoid receptors, A₁ adenosine receptors, α₁ adrenergic receptors, BB₂ bombesin receptors, B₂ bradykinin receptors, calcium-sensing receptors, chemokine receptors, KSHV-ORF74 chemokine receptors, NK₁ tachykinin receptors, thyroid-stimulating hormone (TSH) receptors, protease-activated receptors, neuropeptide receptors, adenosine A2B receptors, P2Y purinoceptors, metabolic glutamate receptors, GRK5, GPCR-30, CCR5, and CXCR4.

In another embodiment, the water-soluble polypeptide retains the at least some of the ligand-binding activity of the membrane protein. In some embodiments, the GPCRs are mammalian receptors.

In a further embodiment, one or more amino acids within potential ligand binding sites of the native membrane protein are not replaced. In an aspect of this embodiment, examples of native membrane proteins with potential ligand-binding sites having one or more amino acids not replaced include: purinergic receptors (P2Y₁, P2Y₂, P2Y₄, P2Y₆), M₁ and M₃ muscarinic acetylcholine receptors, receptors for thrombin [protease-activated receptor (PAR)-1, PAR-2], thromboxane (TXA₂), sphingosine 1-phosphate (S1P₂, S1P₃, S1P₄ and S1P₅), lysophosphatidic acid (LPA₁, LPA₂, LPA₃), angiotensin II (AT₁), serotonin (5-HT₂, and 5-HT₄), somatostatin (sst₅), endothelin (ET_{A} and ET_{B}), cholecystokinin (CCK₁), V₁ₐ vasopressin receptors, D₅ dopamine receptors, fMLP formyl peptide receptors, GAL₂ galanin receptors, EP₃ prostanoid receptors, A₁ adenosine receptors, α₁ adrenergic receptors, BB₂ bombesin receptors, B₂ bradykinin receptors, calcium-sensing receptors, chemokine receptors, KSHV-ORF74 chemokine receptors, NK₁ tachykinin receptors, thyroid-stimulating hormone (TSH) receptors, protease-activated receptors, neuropeptide receptors, adenosine A2B receptors, P2Y purinoceptors, metabolic glutamate receptors, GRK5, GPCR-30, CCR5, and CXCR4.

The invention further encompasses a method of treatment for a disorder or disease that is mediated by the activity of a membrane protein, comprising the use of a subject water-soluble polypeptide to treat said disorders and diseases, wherein said water-soluble polypeptide comprises a modified α-helical domain, with one or more domains involved in ligand binding absent, and wherein said water-soluble polypeptide retains the ligand-binding activity of the native membrane protein. Examples of such disorders and diseases include, but are not limited to, cancer, small cell lung cancer, melanoma, breast cancer, Parkinson's disease, cardiovascular disease, hypertension, and asthma.

As described herein, the water-soluble peptides described herein can be used for the treatment of conditions or diseases mediated by the activity of a membrane protein. In certain aspects, the partial variant water-soluble peptides can act as “decoys” for the membrane receptor and bind to the ligand that activates the membrane receptor. As such, the partial variant water-soluble peptides described herein can be used to reduce the activity of a membrane protein. These partial variant water-soluble peptides can remain in the circulation and bind to specific ligands, thereby reducing the activity of membrane bound receptors. For example, the GPCR CXCR4 is over-expressed in small cell lung cancer and facilitates metastasis of tumor cells. Binding of this ligand by a partial variant water-soluble peptide such as that described herein may significantly reduce metastasis.

The chemokine receptor, CXCR4, is known in viral research as a major co-receptor for the entry of T cell line-tropic HIV (Feng, et al. (1996) Science 272: 872-877; Davis, et al. (1997) J Exp Med 186: 1793-1798; Zaitseva, et al. (1997) Nat Med 3: 1369-1375; Sanchez, et al. (1997) J Biol Chem 272: 27529-27531). T Stromal cell derived factor 1 (SDF-1) is a chemokine that interacts specifically with CXCR4. When SDF-1 binds to CXCR4, CXCR4 activates Gαi protein-mediated signaling (pertussis toxin-sensitive) (Chen, et al. (1998) Mol Pharmacol 53: 177-181), including downstream kinase pathways such as Ras/MAP Kinases and phosphatidylinositol 3-kinase (PI3K)/Akt in lymphocyte, megakaryocytes, and hematopoietic stem cells (Bleul, et al. (1996) Nature 382: 829-833; Deng, et al. (1997) Nature 388: 296-300; Kijowski, et al. (2001) Stem Cells 19: 453-466; Majka, et al. (2001) Folia. Histochem. Cytobiol. 39: 235-244; Sotsios, et al. (1999) J. Immunol. 163: 5954-5963; Vlahakis, et al. (2002) J. Immunol. 169: 5546-5554). In mice transplanted with human lymph nodes, SDF-1 induces CXCR4-positive cell migration into the transplanted lymph node (Blades, et al. (2002) J. Immunol. 168: 4308-4317).

Recently, studies have shown that CXCR4 interactions may regulate the migration of metastatic cells. Hypoxia, a reduction in partial oxygen pressure, is a microenvironmental change that occurs in most solid tumors and is a major inducer of tumor angiogenesis and therapeutic resistance. Hypoxia increases CXCR4 levels (Staller, et al. (2003) Nature 425: 307-311). Microarray analysis on a sub-population of cells from a bone metastatic model with elevated metastatic activity showed that one of the genes increased in the metastatic phenotype was CXCR4. Furthermore, overexpression CXCR4 in isolated cells significantly increased the metastatic activity (Kang, et al. (2003) Cancer Cell 3: 537-549). In samples collected from various breast cancer patients, Muller et al. (Muller, et al. (2001) Nature 410: 50-56) found that CXCR4 expression level is higher in primary tumors relative to normal mammary gland or epithelial cells. Moreover, CXCR4 antibody treatment has been shown to inhibit metastasis to regional lymph nodes when compared to control isotypes that all metastasized to lymph nodes and lungs (Muller, et al. (2001)). As such a decoy therapy model is suitable for treating CXCR4 mediated diseases and disorders.

Another aspect of the invention relates to the treatment of a disease or disorder involving CXCR4-dependent chemotaxis, wherein the disease is associated with aberrant leukocyte recruitment or activation. The disease is selected from the group consisting of arthritis, psoriasis, multiple sclerosis, ulcerative colitis, Crohn's disease, allergy, asthma, AIDS associated encephalitis, AIDS related maculopapular skin eruption, AIDS related interstitial pneumonia, AIDS related enteropathy, AIDS related periportal hepatic inflammation and AIDS related glomerulo nephritis.

In another aspect, the invention relates to the treatment of a disease or disorder selected from arthritis, lymphoma, non-small lung cancer, lung cancer, breast cancer, prostate cancer, multiple sclerosis, central nervous system developmental disease, dementia, Parkinson's disease, Alzheimer's disease, tumor, fibroma, astrocytoma, myeloma, glioblastoma, an inflammatory disease, an organ transplantation rejection, AIDS, HIV-infection or angiogenesis.

The invention also encompasses a pharmaceutical composition comprising the subject partial variant water-soluble polypeptide and a pharmaceutically acceptable carrier or diluent.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Transdermal administration includes percutaneous absorption of the composition through the skin. Transdermal formulations include patches, ointments, creams, gels, salves and the like. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

An “effective amount” refers to that amount of the therapeutic agent that is sufficient to ameliorate of one or more symptoms of a disorder and/or prevent advancement of a disorder, cause regression of the disorder and/or to achieve a desired effect.

The invention will be better understood in connection with the following example, which is intended as an illustration only and not limiting of the scope of the invention. Various changes and modifications to the disclosed embodiments will be apparent to those skilled in the art and such changes and may be made without departing from the spirit of the invention and the scope of the appended claims.

### EXAMPLES

### Example 1 Using the QTY Code to Produce Water-Soluble Olfactory Receptor (OR) Variants

1) Use the QTY (Glutamine/Asparagine, threonine and tyrosine) replacement method to systematically change the 7-transmembrane α-helix hydrophobic residues leucine (L), isoleucine (I), valine (V), and phenylalanine (F) to the hydrophilic residues glutamine (Q)/Asparagine (N), threonine (T) and tyrosine (Y). This method converts the protein from a water-insoluble olfactory receptor to a water-soluble one.

2) Produce and purify milligram quantities of native and bioengineered olfactory receptors using commercial cell-free in vitro translation systems (Invitrogen and Qiagen).

3) Determine the secondary structure of the purified olfactory receptors using circular dichroism (CD).

4) Determine the binding affinity of the native and bioengineered olfactory receptor variants using microscale thermophoresis.

5) Transfect the native and variant OR genes into HEK293 cells, and use calcium influx assays to measure odorant activation of the native and mutant olfactory receptors. These measurements correlate the microscale thermophoresis binding data to functional responses within cells.

6) Systematically screen the native and bioengineered olfactory receptors for crystallizing conditions in the presence and absence of odorants and the presence and absence of detergent.

The above general steps were used to implement the QTY replacement method in order to design a soluble 7-helical bundle olfactory receptor mOR103-15. An innovation of this study is to convert the water-insoluble olfactory receptor mOR103-15 into a water soluble one with about 10.5% specific residues changes (36aa/340aa). The method systematically and selectively changed key residues at the α-helical positions b, c, f that usually face the hydrophilic surface, while maintaining the hydrophobic residues at α-helical positions a, d, e, g. This synthetic biology design method is general and broadly applicable to the study of other olfactory receptors and G-protein coupled receptors. This strategy has the potential to overcome the bottleneck of crystallizing olfactory receptors, as well as additional GPCRs and other membrane proteins. While the design to change the solubility of the sequence is focused on the b,c,f positions of the helical wheel, some further changes to other parts of the sequence can be made without significantly affecting the function or structure of the peptide, polypeptide or protein. For example, conservative mutations can be made.

1) Use of QTY replacements to design a water-soluble 7-helical bundle olfactory receptor mOR103-15. Synthetic biology methods were used to convert a water-insoluble olfactory receptor into a water-soluble one with ˜10.5% of the residues changes (36aa/340aa). The method systematically and selectively changed key residues at the α-helical positions b, c, f (which usually form the hydrophilic surface), but maintained the hydrophobic residues at α-helical positions a, d, e, g.

In this soluble olfactory receptor design, the following substitutions were made: leucine→glutamine (L→Q), isoleucine/valine→threonine (I/V→T) and phenylalanine→tyrosine (F→Y). In the study, the secondary structure of the water-soluble olfactory receptor is examined, and its odorant-binding capabilities are determined. The secondary structure and binding of native olfactory receptor with the designed water-soluble olfactory receptor can be compared. Finally, milligram quantities of the water-soluble receptor are produced, and crystal screens with and without odorants are set up. The sequence below is disclosed as SEQ ID NO: 1.

MERRNHTGRV SEFVLLGFPA PAPQRALQFF QSLQAYVQTL TENIQTITAI RNHPTLHKPM YYFLANMSYL ETWYTTVTTP
abcdefga bcdefgabcd efgabcdefg a a bcdefgabcd afgabcdefg
KMQAGYIGSE ENHGQLISFE ACMTQLYFFQ GLGCTECTLL AVMAYDRYVA TCHPLHYPVI VSSRQCVQMA AGSWAGGFGT
abcdefg abcdefgab cdefgabcde fgabcdefga bc abcdefg abcdefgagc
SMTKVYQISR LSYCGPNTIN HFFCDVSPLL NLSCTDMSTA ELTDFIQAIY TLLGPLSTTG ASYMAITGAV MRIPSAAGRH
defgabcd abcdefgab cdefgabcde fgabcdefga a
KAFSTCASHL TTVITYYAAS IYTYARPKAL SAFDTNKLVS VLYAVITPLQ NPITYCQRNQ EVKKALRRTL HALQGQDANT
bcdefgabcd efgabcdefg abcd abcdef gabcdefgab cdefgabc
KKSSRDGGSS GTETSQVAPA. (36aa mutations/340aa, ~10.5% mutations)

2) Produce and purify milligram quantities of native and bioengineered variants of olfactory receptors. Commercial cell-free systems can be used to produce milligrams of native and water-soluble mORI03-15. The native and variant olfactory receptors can be produced and purified in one day using immunoaffinity purification. Gel filtration can then be used to separate the monomeric and dimeric receptor forms.

3) Determine secondary structure using circular dichroism. Circular dichroism (CD) spectral analysis was then used to measure the secondary structures of the purified receptors. CD is a very sensitive technique that is be able to detect any small structural changes between the native and mutant receptors. Specifically, CD analysis can be used to calculate the percentage of α-helices and β-sheets in a protein. If a proteins' structure is altered, it can be revealed in the CD analysis. In addition to determining whether specific mutations alter receptor structure, CD can also be used to measure any odorant-induced structural changes.

4) Assay ligand-binding of olfactory receptors. Microscale thermophoresis are used to measure the binding affinity of the native and bioengineered proteins and their odorant ligands. The key advantages of this technique over SPR or other ligand binding technologies are that they are totally surface-free and label free. Thus, the receptors do not need to be modified. The measurements can be performed in solution using native tryptophan as a signal source. Additionally, small ligands (MW ˜200 Daltons) can be reliably measured. Furthermore, each measurement needs very small amount of sample, thus, save the precious receptor samples. These results show whether the mutant olfactory receptors are capable of binding odorants as efficiently as the native protein.

5) Use calcium influx activation assay to measure olfactory receptor activation. The calcium influx assays can be used to examine odorant-induced activation of the native and variant olfactory receptors in HEK293 cells. This data can be correlated to the microscale thermophoresis measurements. Microscale thermophoresis directly measures ligand binding, while calcium influx assays measure activation. Combined, these assays verify whether specific mutations affect binding, activation, or both. Additionally, agonist and antagonist ligands can be distinguished.

6) Systematic screen for crystallization conditions. The native and bioengineered variant olfactory receptors can be systematically screened for crystallizing conditions in the absence and presence of odorants. The technology for crystallization screening of water-soluble proteins is well developed. Commercial screens are available which supply a variety of precipitants, salts, buffers with fine tuned pH gradients, and a range of cationic and anionic substances. All of these variables are well known and can be used in crystallizing membrane proteins. An additional unique ingredient of membrane protein screens is the presence of one of more detergent molecules. However, precipitation techniques involving slow water removal from the hanging drop may continue to be effective. Although it is useful to form large crystals, the results of a crystal screen may yield smaller crystals.

The procedures described above can be used in all GPCR variants, including full-length and partial/non-full length variants.

### Example 2 Non-Full Length QTY Variants of CCR5 Binds Natural Ligand Rantes (CCL5)

A library of candidate full length and partial (non-full length) GPCR variants were generated using the QTY substitution method described in U.S. Pat. Nos. 8,637,452, 9,309,302, and US-2015-0370961-A1 (all incorporated herein by reference), based on the CCR5 wildtype sequence. These candidates were then selected using yeast 2-Hybrid screen and subsequent stringent yeast mating selections, namely in vivo selections, to identify candidates that bind to the natural ligand of CCR5—Rantes (CCL5). A list of 8 non-full length and 1 full length CCR5^{QTY} variants were identified, and their sequences listed below.

SZ162a = CCR5QTY(162a) = (5NA-18: Contig1)
Weak gene activation but specific binding
(SEQ ID NO: 2)
MDYQVSSPIYDINYYTSEPCQKINVKQIAARQQPPQYSQTYTFGYTGNMQ
TTQTQINCKRLKSMTDIYLQNQAISDQFFQQTTPFWAHYAAAQWDFGNTM
CQQQTGQYFTGEKFRNYLLVFFQKHIAKRFCKCCSIFQQEAPERASSVYT
RSTGEQEISVGL*
SZ185a = CCR5QTY(185a) = (5CA-4: Contig1)
Medium gene activation but specific binding
(SEQ ID NO: 3)
MDYQVSSPIYDINYYTSEPCQKINVKQIAARQQPPQYSQTFTFGYTNMQT
TQTQTNCKRLKSMTDIYLQNQAISDQYYQFWAPYNTVQQLNTFQEFFGLN
NCSSSNRLDQAMQTTETQGMTHCCTNPIIYAFTGEKFRNYLLVFFQKHIA
KRFCKCCSIFQQEAPERASSVYTRSTGEQEISVGL*
SZ186a = CCR5QTY(186a) = (CC15.D2-4: Contig1)
Weak gene activation but specific binding
(SEQ ID NO: 4)
MDYQVSSPIYDINYYTSEPCQKINVKQIAARQQPPQYSQTFTFGFTGNMQ
TTQTQINCKRLKSMTDIYQQNQATSDQYYQYWAPYNTVQQQNTFQEFFGL
NNCSSSNRLDQAMQVTETQGMTHCCTNPTIYAYVGEKFRNYLLVFFQKHI
AKRFCKCCSIFQQEAPERASSVYTRSTGEQEISVGL*
SZ190a = CCR5QTY(190a) = (CC15.D2-2: Contig1)
Weak gene activation but specific binding
(SEQ ID NO: 5)
MDYQVSSPIYDINYYTSEPCQKINVKQIAARQQPPQYSQTYTFGFTGNMQ
TTQTQINCKRLKSMTDIYLQNQAISDQYFQQTTPYWAPYNTVQQLNTFQE
FFGLNNCSSSNRLDQAMQTTETQGMTHCCINPIIYAFVGEKFRNYLLVFF
QKHIAKRFCKCCSIFQQEAPERASSVYTRSTGEQEISVGL*
SZ190b = CCR5QTY(190b) = (5NA-17: Contig1)
weak gene activation but specific binding
(SEQ ID NO: 6)
MDYQVSSPIYDINYYTSEPCQKINVKQIAARLQPPQYSQTFTFGFTGNMQ
TTQTQINCKRLKSMTDIYLQNQAISDQFFQQTTPYWAPYNTVQQQNTFQE
FFGLNNCSSSNRLDQAMQVTETQGMTHCCTNPTIYAFVGEKFRNYLLVFF
QKHIAKRFCKCCSIFQQEAPERASSVYTRSTGEQEISVGL*
SZ190c = CCR5QTY(190c) = (5NB-6: Contig1)
Medium gene activation and specific binding
(SEQ ID NO: 7)
MDYQVSSPIYDINYYTSEPCQKINVKQIAARLQPPQYSQTFTFGYTGNMQ
VTQTQINCKRLKSMTDIYLQNQAISDQFFQQTTPYWAPYNTVQQQNTFQE
FFGLNNCSSSNRLDQAMQVTETLGMTHCCTNPIIYAYTGEKFRNYLLVFF
QKHIAKRFCKCCSIFQQEAPERASSVYTRSTGEQEISVGL*
SZ190d CCR5QTY(190d) = (5CA-3: Contig1)
Strong gene activation and specific binding
(SEQ ID NO: 8)
MDYQVSSPIYDINYYTSEPCQKINVKQIAARLQPPQYSQTFTFGFTGNMQ
TTQTQINCKRLKSMTDIYLQNQAISDQYYQQTTPYWAPYNTVQQQNTFQE
FFGLNNCSSSNRLDQAMQVTETLGMTHCCTNPIIYAFTGEKFRNYLLVFF
QKHIAKRFCKCCSIFQQEAPERASSVYTRSTGEQEISVGL*
SZ218a = CCR5QTY(218a) = (5NA-43) Strong gene
activation but less specific binding
(SEQ ID NO: 9)
MDYQVSSPIYDINYYTSEPCQKINVKQIAARQQPPQYSQTYTFGFTGNMQ
TTQTQINCKRLKSMTDIYLQNQAISDQFFQQTTPFWAHYAAAQWDFGNTM
CQQQTGQYFTGYYSGTYYTTQQLNTFQEFFGLNNCSSSNRLDQAMQTTET
QGMTHCCINPTTYAYVGEKFRNYLLVFFQKHIAKRFCKCCSIFQQEAPER
ASSVYTRSTGEQEISVGL*
SZ352a = CCR5QTY(352a) = (CC15-22) Not strong
gene activation but specific binding
(SEQ ID NO: 10)
MDYQVSSPIYDINYYTSEPCQKINVKQIAARQQPPQYSQTFTYGFTGNMQ
TTQTQINCKRLKSMTDIYLQNQAISDQYYQQTTPYWAHYAAAQWDFGNTM
CQQQTGQYFTGYYSGTYYTTQQTTDRYLAVVHAVFALKARTTTYGTTTST
TTWTTATYASQPGTTYTRSQKEGLHYTCSSHFPYSQYQFWKNFQTLKITI
QGQVQPQQTMVTCYSGTQKTLLRCRNEKKRHRAVRQTFTTMTTYYQYWAP
YNTTQQLNTFQEFFGLNNCSSSNRLDQAMQVTETQGMTHCCTNPTIYAYV
GEKFRNYLLVFFQKHIAKRFCKCCSIFQQEAPERASSVYTRSTGEQEISV
GL*

Fluorescent surface-free MST (MicroScale Thermophoresis) in vitro binding assay was then used to demonstrate the binding between these partial water-soluble variants of CCR5 (labeled with a dye) and its natural ligand Rantes (CCL5). Each run of assay systematically measures 16 capillaries with identical concentration of a non-full length CCR5^{QTY} variant, but different concentrations of Rantes (Prospecbio http://www.prospecbio.com/Rantes/) with 2 duplicate measurements (Technical repeat 1 and 2).

Ligand biding measurements were run for 3 of the 8 variants, 1 full length (SZ352a) as control, and 2 non-full length variants SZ218a and SZ190b.

Briefly, serial dilution of the target molecule Rantes was prepared in buffer to match the final buffer conditions in the reaction mix. The highest concentration of target was 6.0 μM, and the lowest 184.0 pM. About 5 μl of each dilution step were mixed with 5 μl of the fluorescent molecule—a partial water-soluble QTR variant of CCR5 (e.g., CCR5-SZ218a). The final reaction mixture, which was filled in capillaries, contained a respective amount of target molecule (max. conc. 3.0 μM, min conc. 92.0 pM) and constant 5 nM fluorescent molecule.

The samples were analyzed on a Monolith NT.115 Pico at 25° C., with 12% LED power and 80% Laser power. No sticking of the fluorescent interaction partner to the capillary walls was indicated in the capillary scan.

Certain detailed assay conditions for SZ218a were listed below:

| | |
|---|---|
| Fluorescent Molecule | |
| Name: | CCR5_SZ218a DY647P1 |
| Concentration (constant): | 5 nM |
| Vol. in final reaction mix: | 5 μl |
| Target Molecule | |
| Name: | Rantes |
| Max. concentration: | 3 μM |
| Min. concentration: | 92 pM |
| Vol. in final reaction mix: | 5 μl |
| Buffer Conditions*: | 1x PBS pH 7.4, 5 mM DTT |
| Dilution Steps: | 1:1 |
| Capillary type**: | Premium coated |
| MST Instrument: | Monolith NT.115 Pico |
| Laser Power: | 80% |
| LED Power: | 12% |
| Temperature: | 25° C. |
| Analysis Method: | Thermophoresis |
| Interaction: | binding |

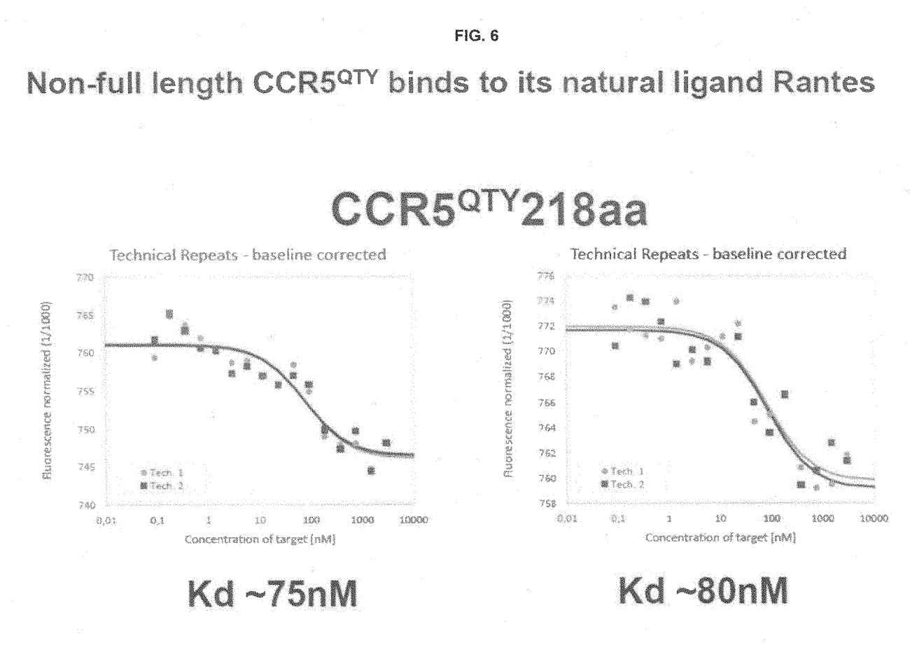

In one run for SZ218a, the two Technical Repeats produced a K_{d} value of 74.5 and 76.2 nM (or about 75 nM). In another run, the measured K_{d} values were 80.2 and 83.2 nM (or about 80 nM). The results are shown in FIG. 6. In a 3^{rd} run in which LED Power was changed from 12% to 15%, the measured K_{d} values were 86.9 and 94.6 nM (data not shown).

Similarly, certain detailed assay conditions for SZ190b were listed below:

| | |
|---|---|
| Fluorescent Molecule | |
| Name; | CCR5_SZ190b DY647P1 |
| Concentration (constant): | 5 nM |
| Vol. in final reaction mix: | 5 μl |
| Target Molecule | |
| Name: | Rantes |
| Max. concentration: | 3 μM |
| Min. concentration: | 92 pM |
| Vol. in final reaction mix: | 5 μl |
| Buffer Conditions*: | 1x PBS pH 7.4, 5 mM DTT |
| Dilution Steps: | 1:1 |
| Capillary type**: | Premium coated |
| MST Instrument: | Monolith NT.115 Pico |
| Laser Power: | 80% |
| LED Power: | 7% |
| Temperature: | 25° C. |
| Analysis Method: | Thermophoresis |
| Interaction: | binding |

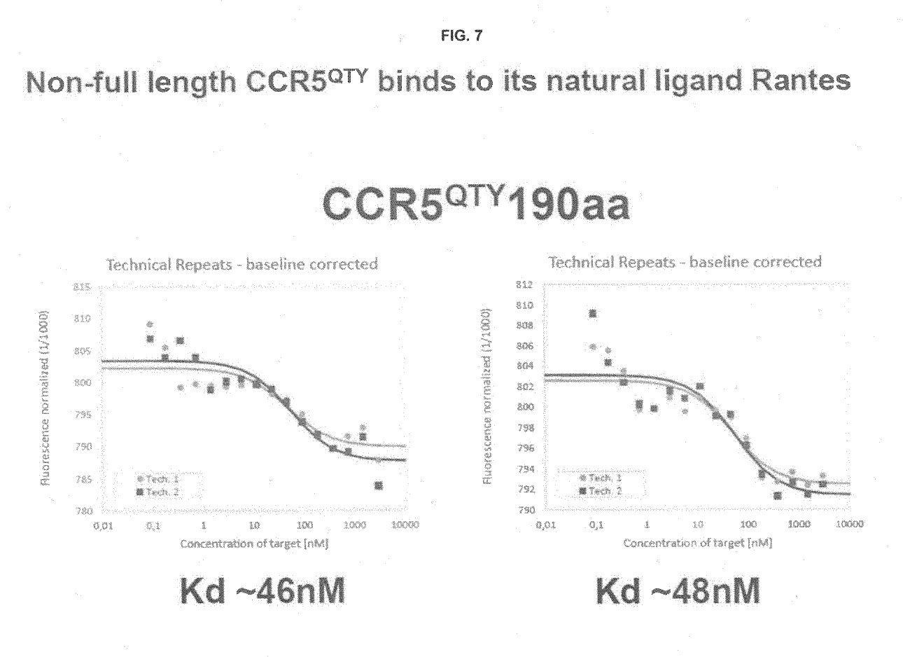

In one run for SZ218a, the two Technical Repeats produced a K_{d} value of 46.5 and 51.8 nM. In another run, the measured K_{d} values were 27.4 and 56.5 nM (or about 80 nM). In a 3^{rd} run, the measured K_{d} values were 32.0 and 54.9 nM (data not shown). See FIG. 7.

The ranking order of binding affinity for the variants is: SZ218a>SZ190d>SZ190c>SZ190b>SZ162a>SZ185a>SZ186a>SZ190a.

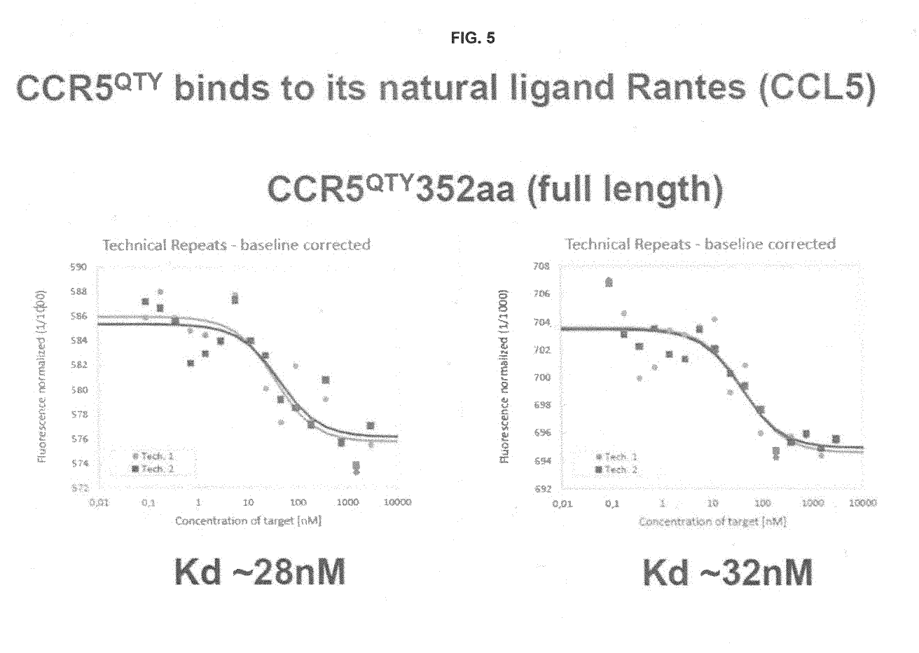

As a comparison, the measured K_{d} values for the full length CCR5^{QTY} variant were about 28-32 nM (FIG. 5).

While this invention has been particularly shown and described with references to preferred embodiments thereof, it should be understood by those skilled in the art that various changes in form and details may be made therein without departing from the scope of the invention encompassed by the appended claims.

1. A water-soluble variant of a G-protein coupled receptor (GPCR), wherein in said variant:
(1) 7-transmembrane α-helical hydrophobic residues Leucine (L), isoleucine (I), valine (V), and phenylalanine (F) in hydrophilic surface α-helical positions b, c, and f but not positions a, d, e, and g of the GPCR have been substituted by glutamine (Q) or Asparagine (N), threonine (T), threonine (T), and tyrosine (Y), respectively, and,
(2) a domain of the GPCR, or a portion of the domain, is absent, wherein said domain is selected from the group consisting of: the N-terminal extracellular sequence, a 7-transmembrane α-helical domain, and an extracellular loop (EC).

2. The variant of claim 1, wherein the variant has a biological activity of the GPCR.

3. The variant of claim 2, wherein the biological activity is ligand binding.

4. The variant of claim 3, wherein the biological activity is at least substantially similar binding affinity for a native ligand of the GPCR.

5. The variant of claim 1, wherein the pI of the variant is substantially the same as the pI of the GPCR.

6. The variant of claim 1, wherein said variant comprises conservative substitutions at other parts of the variant.

7. The variant of claim 1, wherein at least 25 said 7-transmembrane α-helical hydrophobic residues L, I, V, and F are replaced.

8. The variant of claim 1, wherein the GPCR is a mammalian receptor.

9. (canceled)

10. The variant of claim 1, wherein the GPCR is a CXCR4 or CCR5.

11. The variant of claim 1, comprising the full length or a partial N-terminal extracellular sequence.

12. The variant of claim 1, wherein a 7-transmembrane α-helical domain is absent.

13. The variant of claim 12, wherein two or more 7-transmembrane α-helical domains are absent.

14-15. (canceled)

16. The variant of claim 1, wherein an EC is absent.

17. The variant of claim 16, wherein two or more ECs are absent.

18-19. (canceled)

20. The variant of claim 1, wherein an IC is absent.

21. The variant of claim 20, wherein two or more ICs are absent.

22-23. (canceled)

24. The variant of claim 1, wherein the variant has reduced ligand binding affinity as compared to the wildtype GPCR.

25. The variant of claim 1, wherein the variant has reduced ligand binding specificity as compared to the wildtype GPCR

26. (canceled)

27. A method for treating a mammal suffering from a disorder or disease that is mediated by the activity of a GPCR polypeptide, comprising administering to said mammal an effective amount of the water-soluble variant of claim 1.

28. A pharmaceutical composition comprising an effective amount of a variant of claim 1, and a pharmaceutically acceptable diluent or carrier.


SEQUENCE LISTING (22 sequences)

SEQ ID NO: 1 - protein, 340 aa, Artificial Sequence
  Description of Artificial Sequence Synthetic polypeptide
MERRNHTGRVSEFVLLGFPAPAPQRALQFFQSLQAYVQTLTENIQTITAIRNHPTLHKPMYYFLANMSYLETWYTTVTTPKMQAGYIGSEENHGQLISFEACMTQLYFFQGLGCTECTLLAVMAYDRYVATCHPLHYPVIVSSRQCVQMAAGSWAGGFGTSMTKVYQISRLSYCGPNTINHFFCDVSPLLNLSCTDMSTAELTDFIQAIYTLLGPLSTTGASYMAITGAVMRIPSAAGRHKAFSTCASHLTTVITYYAASIYTYARPKALSAFDTNKLVSVLYAVITPLQNPIIYCQRNQEVKKALRRTLHLAQGQDANTKKSSRDGGSSGTETSQVAPA

SEQ ID NO: 2 - protein, 162 aa, Artificial Sequence
  Description of Artificial Sequence Synthetic polypeptide
MDYQVSSPIYDINYYTSEPCQKINVKQIAARQQPPQYSQTYTFGYTGNMQTTQTQINCKRLKSMTDIYLQNQAISDQFFQQTTPFWAHYAAAQWDFGNTMCQQQTGQYFTGEKFRNYLLVFFQKHIAKRFCKCCSIFQQEAPERASSVYTRSTGEQEISVGL

SEQ ID NO: 3 - protein, 185 aa, Artificial Sequence
  Description of Artificial Sequence Synthetic polypeptide
MDYQVSSPIYDINYYTSEPCQKINVKQIAARQQPPQYSQTFTFGYTNMQTTQTQTNCKRLKSMTDIYLQNQAISDQYYQFWAPYNTVQQLNTFQEFFGLNNCSSSNRLDQAMQTTETQGMTHCCTNPIIYAFTGEKFRNYLLVFFQKHIAKRFCKCCSIFQQEAPERASSVYTRSTGEQEISVGL

SEQ ID NO: 4 - protein, 186 aa, Artificial Sequence
  Description of Artificial Sequence Synthetic polypeptide
MDYQVSSPIYDINYYTSEPCQKINVKQIAARQQPPQYSQTFTFGFTGNMQTTQTQINCKRLKSMTDIYQQNQATSDQYYQYWAPYNTVQQQNTFQEFFGLNNCSSSNRLDQAMQVTETQGMTHCCTNPTIYAYVGEKFRNYLLVFFQKHIAKRFCKCCSIFQQEAPERASSVYTRSTGEQEISVGL

SEQ ID NO: 5 - protein, 190 aa, Artificial Sequence
  Description of Artificial Sequence Synthetic polypeptide
MDYQVSSPIYDINYYTSEPCQKINVKQIAARQQPPQYSQTYTFGFTGNMQTTQTQINCKRLKSMTDIYLQNQAISDQYFQQTTPYWAPYNTVQQLNTFQEFFGLNNCSSSNRLDQAMQTTETQGMTHCCINPIIYAFVGEKFRNYLLVFFQKHIAKRFCKCCSIFQQEAPERASSVYTRSTGEQEISVGL

SEQ ID NO: 6 - protein, 190 aa, Artificial Sequence
  Description of Artificial Sequence Synthetic polypeptide
MDYQVSSPIYDINYYTSEPCQKINVKQIAARLQPPQYSQTFTFGFTGNMQTTQTQINCKRLKSMTDIYLQNQAISDQFFQQTTPYWAPYNTVQQQNTFQEFFGLNNCSSSNRLDQAMQVTETQGMTHCCTNPTIYAFVGEKFRNYLLVFFQKHIAKRFCKCCSIFQQEAPERASSVYTRSTGEQEISVGL

SEQ ID NO: 7 - protein, 190 aa, Artificial Sequence
  Description of Artificial Sequence Synthetic polypeptide
MDYQVSSPIYDINYYTSEPCQKINVKQIAARLQPPQYSQTFTFGYTGNMQVTQTQINCKRLKSMTDIYLQNQAISDQFFQQTTPYWAPYNTVQQQNTFQEFFGLNNCSSSNRLDQAMQVTETLGMTHCCTNPIIYAYTGEKFRNYLLVFFQKHIAKRFCKCCSIFQQEAPERASSVYTRSTGEQEISVGL

SEQ ID NO: 8 - protein, 190 aa, Artificial Sequence
  Description of Artificial Sequence Synthetic polypeptide
MDYQVSSPIYDINYYTSEPCQKINVKQIAARLQPPQYSQTFTFGFTGNMQTTQTQINCKRLKSMTDIYLQNQAISDQYYQQTTPYWAPYNTVQQQNTFQEFFGLNNCSSSNRLDQAMQVTETLGMTHCCTNPIIYAFTGEKFRNYLLVFFQKHIAKRFCKCCSIFQQEAPERASSVYTRSTGEQEISVGL

SEQ ID NO: 9 - protein, 218 aa, Artificial Sequence
  Description of Artificial Sequence Synthetic polypeptide
MDYQVSSPIYDINYYTSEPCQKINVKQIAARQQPPQYSQTYTFGFTGNMQTTQTQINCKRLKSMTDIYLQNQAISDQFFQQTTPFWAHYAAAQWDFGNTMCQQQTGQYFTGYYSGTYYTTQQLNTFQEFFGLNNCSSSNRLDQAMQTTETQGMTHCCINPTTYAYVGEKFRNYLLVFFQKHIAKRFCKCCSIFQQEAPERASSVYTRSTGEQEISVGL

SEQ ID NO: 10 - protein, 352 aa, Artificial Sequence
  Description of Artificial Sequence Synthetic polypeptide
MDYQVSSPIYDINYYTSEPCQKINVKQIAARQQPPQYSQTFTYGFTGNMQTTQTQINCKRLKSMTDIYLQNQAISDQYYQQTTPYWAHYAAAQWDFGNTMCQQQTGQYFTGYYSGTYYTTQQTTDRYLAVVHAVFALKARTTTYGTTTSTTTWTTATYASQPGTTYTRSQKEGLHYTCSSHFPYSQYQFWKNFQTLKITIQGQVQPQQTMVTCYSGTQKTLLRCRNEKKRHRAVRQTFTTMTTYYQYWAPYNTTQQLNTFQEFFGLNNCSSSNRLDQAMQVTETQGMTHCCTNPTIYAYVGEKFRNYLLVFFQKHIAKRFCKCCSIFQQEAPERASSVYTRSTGEQEISVGL

SEQ ID NO: 11 - protein, 146 aa, Artificial Sequence
  Description of Artificial Sequence Synthetic polypeptide
MEGISIYTSDNYTEEMGSGDYDSMKEPCFREENANFNKTYQPTTYSTTYQTGTSTDSFILLEIIKQGCEFENTVHKWISITEAQAYFHCCQNPTLYAFLGAKFKTSAQHALTSVSRGSSLKILSKGKRGGHSSVSTESESSSFHSS

SEQ ID NO: 12 - protein, 146 aa, Artificial Sequence
  Description of Artificial Sequence Synthetic polypeptide
MEGISIYTSDNYTEEMGSGDYDSMKEPCFREENANFNKIFQPTTYSTTYQTGTSTDSFILLEIIKQGCEFENTVHKWISITEAQAFFHCCLNPIQYAFLGAKFKTSAQHALTSVSRGSSLKILSKGKRGGHSSVSTESESSSFHSS

SEQ ID NO: 13 - protein, 146 aa, Artificial Sequence
  Description of Artificial Sequence Synthetic polypeptide
MEGISIYTSDNYTEEMGSGDYDSMKEPCFREENANFNKIFQPTTYSTTYQTGTSTDSFILLEIIKQGCEFENTVHKWISITEAQAFYHCCLNPIQYAFLGAKFKTSAQHALTSVSRGSSLKILSKGKRGGHSSVSTESESSSFHSS

SEQ ID NO: 14 - protein, 146 aa, Artificial Sequence
  Description of Artificial Sequence Synthetic polypeptide
MEGISIYTSDNYTEEMGSGDYDSMKEPCFREENANFNKIFQPTTYSTTFQTGTSTDSFILLEIIKQGCEFENTVHKWISITEAQAFYHCCLNPIQYAFLGAKFKTSAQHALTSVSRGSSLKILSKGKRGGHSSVSTESESSSFHSS

SEQ ID NO: 15 - protein, 146 aa, Artificial Sequence
  Description of Artificial Sequence Synthetic polypeptide
MEGISIYTSDNYTEEMGSGDYDSMKEPCFREENANFNKTYQPTTYSTTYQTGTSTDSFILLEIIKQGCEFENTVHKWISITEAQAFYHCCLNPIQYAFLGAKFKTSAQHALTSVSRGSSLKILSKGKRGGHSSVSTESESSSFHSS

SEQ ID NO: 16 - protein, 146 aa, Artificial Sequence
  Description of Artificial Sequence Synthetic polypeptide
MEGISIYTSDNYTEEMGSGDYDSMKEPCFREENANFNKTYQPTTYSTTYQTGTSTDSFILLEIIKQGCEFENTVHKWISITEAQAFFHCCLNPIQYAFLGAKFKTSAQHALTSVSRGSSLKILSKGKRGGHSSVSTESESSSFHSS

SEQ ID NO: 17 - protein, 146 aa, Artificial Sequence
  Description of Artificial Sequence Synthetic polypeptide
MEGISIYTSDNYTEEMGSGDYDSMKEPCFREENANFNKIFQPTTYSTTYQTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 18 - protein, 146 aa, Artificial Sequence
  Description of Artificial Sequence Synthetic polypeptide
MEGISIYTSDNYTEEMGSGDYDSMKEPCFREENANFNKIFQPTTYSTTFQTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 19 - protein, 146 aa, Artificial Sequence
  Description of Artificial Sequence Synthetic polypeptide
MEGISIYTSDNYTEEMGSGDYDSMKEPCFREENANFNKIFQPTTYSTTFQTGTSTDSFILLEIIKQGCEFENTVHKWISTTEALAYFHCCQNPTQYAFLGAKFKTSAQHALTSVSRGSSLKILSKGKRGGHSSVSTESESSSFHSS

SEQ ID NO: 20 - protein, 146 aa, Artificial Sequence
  Description of Artificial Sequence Synthetic polypeptide
MEGISIYTSDNYTEEMGSGDYDSMKEPCFREENANFNKIFQPTTYSTTYQTGTSTDSFILLEIIKQGCEFENTVHKWISTTEALAYYHCCLNPIQYAFLGAKFKTSAQHALTSVSRGSSLKILSKGKRGGHSSVSTESESSSFHSS

SEQ ID NO: 21 - protein, 146 aa, Artificial Sequence
  Description of Artificial Sequence Synthetic polypeptide
MEGISIYTSDNYTEEMGSGDYDSMKEPCFREENANFNKIFQPTTYSTTYQTGTSTDSFILLEIIKQGCEFENTVHKWISTTEALAYYHCCQNPTQYAFLGAKFKTSAQHALTSVSRGSSLKILSKGKRGGHSSVSTESESSSFHSS

SEQ ID NO: 22 - protein, 146 aa, Artificial Sequence
  Description of Artificial Sequence Synthetic polypeptide
MEGISIYTSDNYTEEMGSGDYDSMKEPCFREENANFNKIFLPTTYSTTYQTGTSTDSFILLEIIKQGCEFENTVHKWISTTEALAYYHCCQNPTQYAFLGAKFKTSAQHALTSVSRGSSLKILSKGKRGGHSSVSTESESSSFHSS